(19) United States
(12) Patent Application Publication
(10) Pub. No.: US 2025/0031970 A1
(43) Pub. Date: Jan. 30, 2025

(54) INTRAORAL OCT APPARATUS

(71) Applicant: Dental Imaging Technologies Corporation, Quakertown, PA (US)

(72) Inventors: Victor C. WONG, Pittsford, NY (US); Chuanmao FAN, Rochester, NY (US)

(21) Appl. No.: 18/704,246

(22) PCT Filed: Oct. 25, 2022
(86) PCT No.: PCT/US2022/047674

§ 371 Date: Apr. 24, 2024

Related U.S. Application Data
(60) Provisional application No. 63/273,678, filed on Oct. 29, 2021.

(51) Int. Cl. A61B 5/00; G01B 9/02091

(52) U.S. Cl. CPC A61B 5/0066; A61B 5/0088; G01B 9/02091

(57) ABSTRACT

A handheld optical apparatus for imaging a sample has an interferometer having at least output and collection waveguides formed on a photonic integrated circuit substrate. A light source generates light of wavelengths above a threshold wavelength. A first signal detector obtains an interference signal from the interferometer between a first portion of the light scattered from the sample and a reference portion of the light. A processor is programmed with instructions that perform optical coherence tomography processing on the obtained interference signal.

### FIELD OF THE INVENTION

The disclosure relates generally to apparatus for optical coherence tomography (OCT) imaging and more particularly to handheld apparatus that provide OCT capability in a highly compact OCT scanner.

### BACKGROUND OF THE INVENTION

Optical coherence tomography (OCT) is a non-invasive imaging technique that employs interferometric principles to obtain high resolution, cross-sectional tomographic images that characterize the depth structure of a sample. Particularly suitable for in vivo imaging of human tissue, OCT has shown its usefulness in a range of biomedical research and medical imaging applications, such as in ophthalmology, dermatology, oncology, and other fields, as well as in ear-nose-throat (ENT) and dental imaging.

OCT has been described as a type of “optical ultrasound”, imaging reflected energy from within living tissue to obtain cross-sectional data. In an OCT imaging system, light from a wide-bandwidth source, such as a super luminescent diode (SLD) or other light source, is directed along two different optical paths: a reference arm of known length and a sample arm that illuminates the tissue or other subject under study. Reflected and back-scattered light from the reference and sample arms is then recombined in the OCT apparatus and interference effects are used to determine characteristics of the surface and near-surface underlying structure of the sample. Interference data can be acquired by rapidly scanning the illumination across the sample. At each of several thousand points, the OCT apparatus obtains an interference profile which can be used to reconstruct an A-scan with an axial depth into the material that is a factor of light source coherence. For most tissue imaging applications, OCT uses broadband illumination sources and can provide image content at depths of a few millimeters (mm).

Initial OCT apparatus employed a time-domain (TD-OCT) architecture in which depth scanning is achieved by rapidly changing the length of the reference arm using some type of mechanical mechanism, such as a piezoelectric actuator, for example. TD-OCT methods use point-by-point scanning, requiring that the illumination probe be moved or scanned from one position to the next during the imaging session. More recent OCT apparatus use a Fourier-domain architecture (FD-OCT) that discriminates reflections from different depths according to the optical frequencies of the signals they generate. FD-OCT methods simplify or eliminate axial scan requirements by collecting information from multiple depths simultaneously and offer improved acquisition rate and signal-to-noise ratio (SNR). There are two implementations of Fourier-domain OCT: spectral domain OCT (SD-OCT) and swept-source OCT (SS-OCT).

SD-OCT imaging can be accomplished by illuminating the sample with a broadband source and dispersing the reflected and scattered light with a spectrometer onto an array detector, such as a CCD (charge-coupled device) detector, for example. SS-OCT imaging illuminates the sample with a rapid wavelength-tuned laser and collects light reflected during a wavelength sweep using only a single photodetector or balanced photodetector. With both SD-OCT and SS-OCT, a profile of scattered light reflected from different depths is obtained by operating on the recorded interference signals using Fourier transforms, such as Fast-Fourier transforms (FFT), well known to those skilled in the signal analysis arts.

Because of their potential to achieve higher performance at lower cost, FD-OCT systems based on swept-frequency laser sources have attracted significant attention for medical applications that require subsurface imaging in highly scattering tissues.

One of the challenges to SS-OCT is providing a suitable light source that can generate the needed sequence of wavelengths in rapid succession. To meet this need, swept-source OCT systems conventionally employ a high-speed wavelength sweeping laser that is equipped with an intracavity monochrometer or uses some type of external cavity narrowband wavelength scanning filter for tuning laser output. Examples of external devices that have been used for this purpose include a tunable Fabry-Perot filter whose cavity length is adjusted to provide a linear change of longitudinal mode, and a polygon scanner filter that selectively reflects dispersive wavelength light. Fourier domain mode locking is a recently reported technique that has been used to generate a sweeping frequency, generally most useful for OCT imaging using broadband near infrared (BNIR) wavelengths.

Difficulties in adapting OCT for use in intraoral imaging include integrating various modules within the OCT scanner system and managing and coordinating the generation, delivery, sensing, and interpretation of the light signals obtained in the OCT scan. For more widespread OCT acceptance and use, there is a need for more compact component packaging and for OCT configurations that can be readily configured, without complex setup considerations and without cumbersome signal cabling and restrictions on manipulation and movement of the intraoral scanning camera.

### SUMMARY OF THE INVENTION

An aspect of this application is to advance the dental diagnostic imaging or to address the need for more compact OCT instrumentation for intraoral use.

Another aspect of this application is to address, in whole or in part, at least the foregoing and other deficiencies in the related art.

It is another aspect of this application to provide, in whole or in part, at least the advantages described herein.

These objects are given only by way of illustrative example, and such objects may be exemplary of one or more embodiments of the invention. Other desirable objectives and advantages inherently achieved by the disclosed methods may occur or become apparent to those skilled in the art. The invention is defined by the appended claims.

According to an aspect of this application, there is provided a handheld optical apparatus for imaging a sample that can include an interferometer having at least output and collection waveguides formed on a photonic integrated circuit substrate; a light source that generates light of wavelengths above a threshold wavelength; a first signal detector that obtains an interference signal from the interferometer between a first portion of the light scattered from the sample and a reference portion of the light; and a processor that is programmed with instructions that perform optical coherence tomography processing on the obtained interference signal.

According to an aspect of this application, there is provided a handheld intraoral optical imaging apparatus that can include a probe, the probe including an interferometer formed on a photonic integrated circuit substrate, where the interferometer comprises a light source to generate light of wavelengths above a threshold wavelength, an output waveguide, and a collection waveguide; a signal detector to obtain an interference signal from the interferometer between a first portion of the light scattered from an intraoral feature and a reference portion of the light; and a processor that is programmed with instructions that perform optical coherence tomography processing on the obtained interference signal.

### BRIEF DESCRIPTION OF THE DRAWINGS

The foregoing and other objects, features, and advantages of the invention will be apparent from the following more particular description of the embodiments of the invention, as illustrated in the accompanying drawings.

The elements of the drawings are not necessarily to scale relative to each other. Some exaggeration may be necessary in order to emphasize basic structural relationships or principles of operation. Some conventional components that would be needed for implementation of the described embodiments, such as support components used for providing power, for packaging, and for mounting and protecting system optics, for example, are not shown in the drawings in order to simplify description.

FIG. 1A is a schematic diagram showing a related art swept-source OCT (SS-OCT) apparatus using a programmable filter that uses a Mach-Zehnder interferometer.

FIG. 1B is a schematic diagram showing a related art swept-source OCT (SS-OCT) apparatus using a programmable filter that uses a Michelson interferometer.

FIG. 1C is a schematic diagram showing a related art OCT apparatus using a spectrometer in a spectral domain (SD) OCT apparatus.

FIG. 1D is a schematic diagram that shows components of a related art OCT apparatus for FMCW interferometry measurement.

FIG. 1E is a schematic diagram showing a related art interferometer of an FMCW image acquisition apparatus with a Mach-Zehnder configuration.

FIG. 1F is a schematic diagram showing a related art FMCW interferometer with a Michelson configuration.

FIG. 2 is a schematic diagram that shows components of a related art intraoral OCT imaging system.

FIG. 3 shows galvo mirrors used to provide a 2-D scan as part of a related art OCT imaging system probe.

FIG. 4A is a diagram that shows a schematic representation of scanning operation for obtaining a B-scan.

FIG. 4B is a diagram that shows an OCT scanning pattern for C-scan acquisition.

FIGS. 5A-5E are diagrams that show different types of imaging content acquired and generated as part of an OCT processing sequence, using the example of a tooth image having a severe cavity.

FIG. 6 is a schematic diagram that shows a probe configured for OCT imaging according to an example embodiment of the present disclosure.

FIG. 7 is a schematic diagram that shows a probe configured for OCT imaging and employing a fiber array unit according to an example embodiment of the present disclosure.

FIG. 8 is a schematic diagram that shows a probe configured for OCT imaging and having a swept-source light source integrated on a silicon substrate according to an example embodiment of the present disclosure.

FIG. 9 is a schematic diagram that shows a probe configured for spectral-domain OCT imaging using an external light source according to an example embodiment of the present disclosure.

FIG. 10 is a schematic diagram that shows a probe configured for spectral-domain OCT imaging with an on-board spectrometer according to an example embodiment of the present disclosure.

FIG. 11 is a schematic diagram showing a probe configuration that combines OCT scanning and reflectance image acquisition capabilities according to an example embodiment of the present disclosure.

FIG. 12 is a schematic diagram showing a probe configuration for OCT imaging that is battery powered and a wireless transmitter for un-tethered operation according to an example embodiment of the present disclosure.

FIG. 13 is a schematic diagram that shows an intraoral OCT imaging apparatus according to a tethered embodiment according to an example embodiment of the present disclosure.

FIG. 14 is a schematic diagram that shows an intraoral OCT imaging apparatus according to a wireless embodiment according to an example embodiment of the present disclosure.

### DESCRIPTION OF EMBODIMENTS

The following is a description of exemplary embodiments, reference being made to the drawings in which the same reference numerals identify the same elements of structure in each of the several figures.

Where they are used in the context of the present disclosure, the terms “first”, “second”, and so on, do not necessarily denote any ordinal, sequential, or priority relation, but are simply used to more clearly distinguish one step, element, or set of elements from another, unless specified otherwise.

The term “exemplary” indicates that the description is used as an example, rather than implying that it is an ideal.

As used herein, the term “energizable” relates to a device or set of components that perform an indicated function upon receiving power and, optionally, upon receiving an enabling signal.

In the context of the present disclosure, the term “optics” is used generally to refer to lenses and other refractive, diffractive, and reflective components or apertures used for shaping and orienting a light beam. An individual component of this type is termed an optic.

In the context of the present disclosure, the term “scattered light” is used generally to include light that is reflected and backscattered from an object.

In the context of the present disclosure, the terms “viewer”, “operator”, and “user” are considered to be equivalent and refer to the viewing practitioner, technician, or other person who may operate a camera or scanner and may also view and manipulate an image, such as a dental image, on a display monitor. An “operator instruction” or “viewer instruction” is obtained from explicit commands entered by the viewer, such as by clicking a button on the camera or scanner or by using a computer mouse or by touch screen or keyboard entry.

In the context of the present disclosure, the phrase “in signal communication” indicates that two or more devices and/or components are capable of communicating with each other via signals that travel over some type of signal path. Signal communication may be wired or wireless. The signals may be communication, power, data, or energy signals. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

In the context of the present disclosure, the term “camera” relates to a device that is enabled to acquire a reflectance, 2-D digital image from reflected visible or NIR light, such as structured light that is reflected from the surface of teeth and supporting structures.

The general term “scanner” relates to an optical system that projects a scanned light beam of broadband near-IR (BNIR) light that is directed to the tooth surface through a sample arm and acquired, as scattered light returned in the sample arm, for detecting interference with light from a reference arm used in OCT imaging of a surface. The term “raster scanner” relates to the combination of hardware components that scan light toward a sample, as described in more detail subsequently.

The term “subject” refers to the tooth or other portion of a patient that is being imaged and, in optical terms, can be considered equivalent to the “object” of the corresponding imaging system.

In the context of the present disclosure, the phrase “broadband light emitter” refers to a light source that emits a continuous spectrum output over a range of wavelengths at any given point of time. Short-coherence or low-coherence, broadband light sources can include, for example, super luminescent diodes, short-pulse lasers, many types of white-light sources, and supercontinuum light sources. Most short coherence length sources of these types have a coherence length on the order of tens of microns or less.

In the context of the present disclosure, the term “oblique” describes an angular orientation that is not an integer multiple of 90 degrees. Two lines or light paths can be considered to be oblique with respect to each other, for example, if they diverge from or converge toward each other at an angle that is about 5 degrees or more away from parallel, or about 5 degrees or more away from orthogonal.

In the context of the present disclosure, two wavelengths can be considered to be “near” each other when within no more than +/−10 nm apart.

According to an embodiment of the present disclosure, there is provided a programmable light source that can provide variable wavelength illumination. The programmable light source can be used as a swept-source for scanned SS-OCT and other applications that benefit from a controllably changeable spectral pattern.

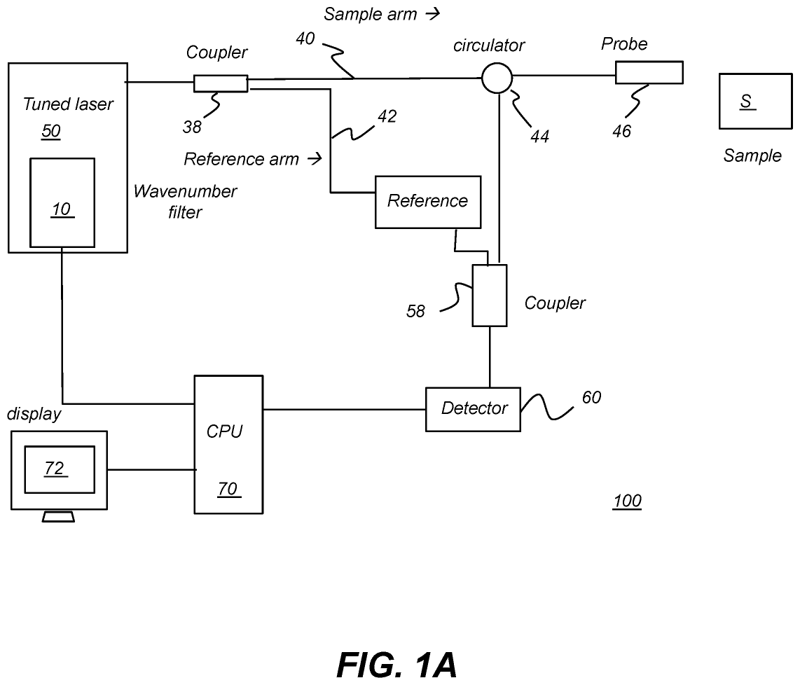

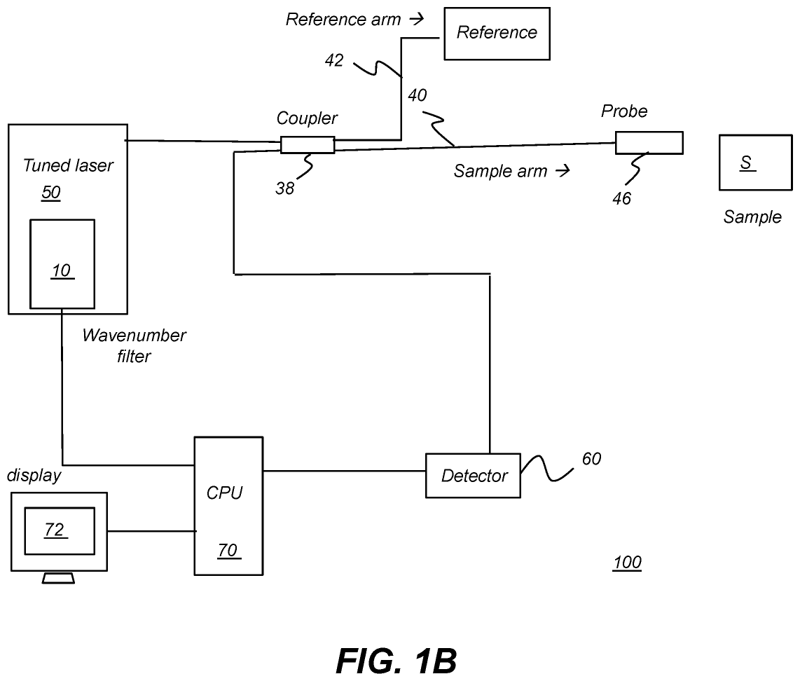

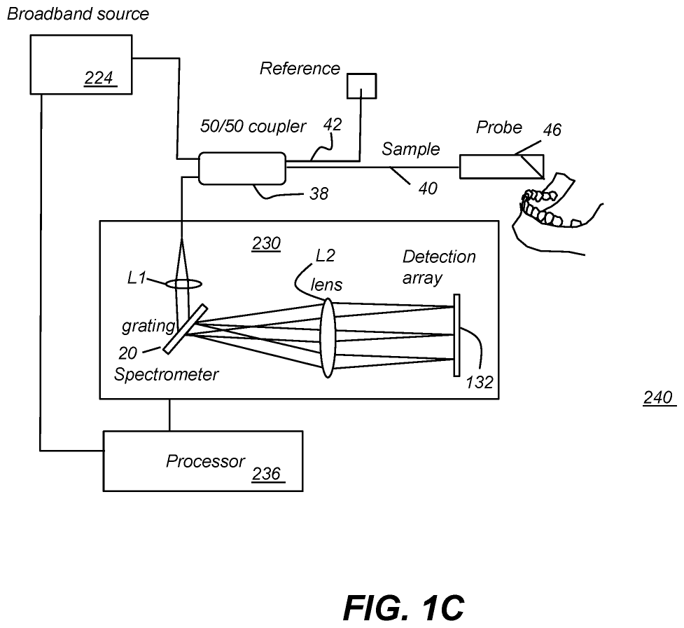

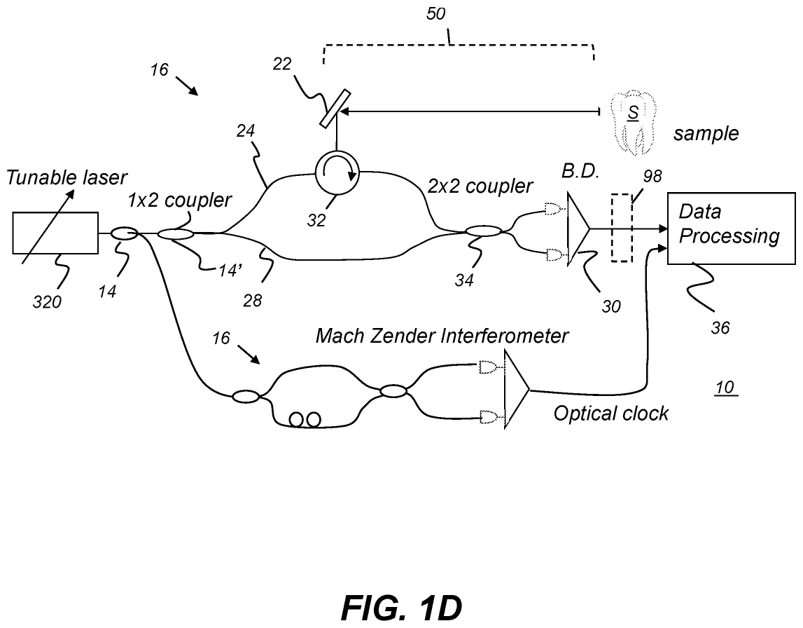

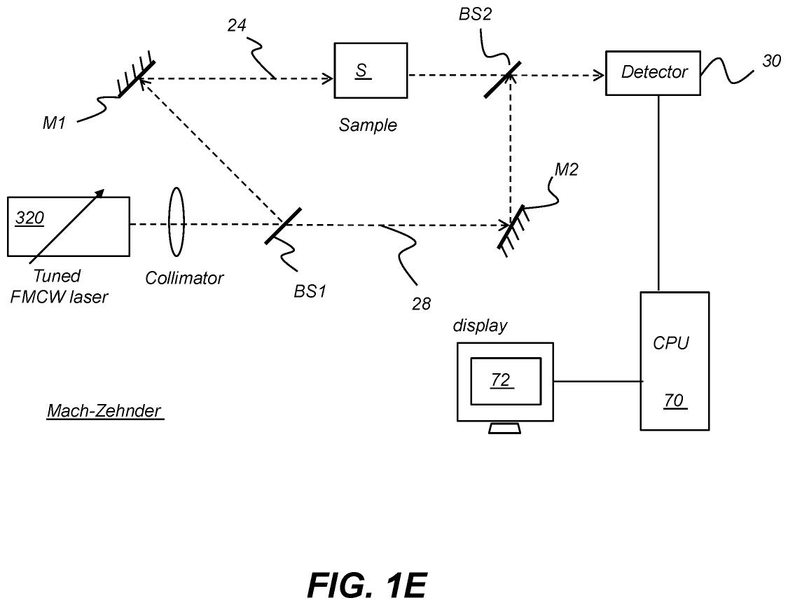

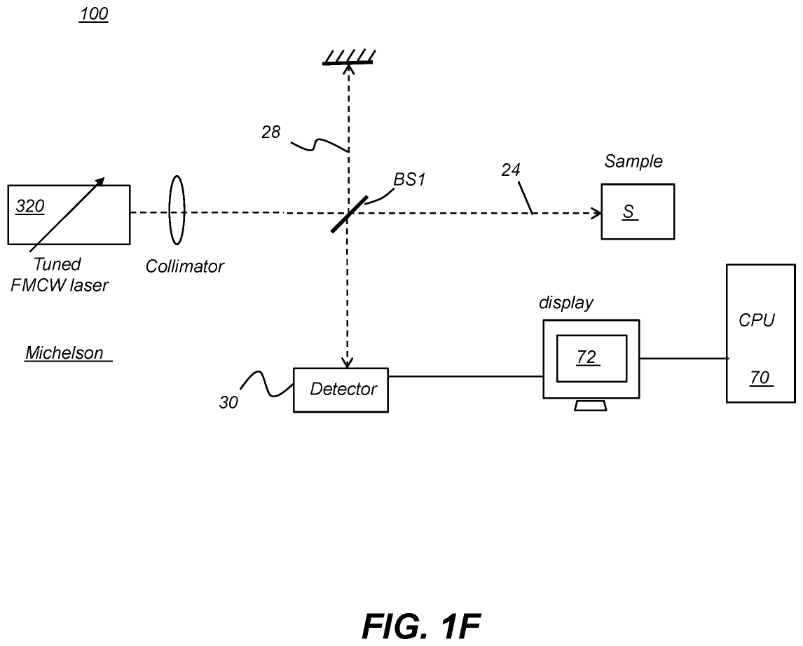

The simplified schematic diagrams of FIGS. 1A and 1B each show the components of a related art swept-source OCT (SS-OCT) apparatus 100 using a programmable filter 10 that is part of a tuned laser 50.

In the FIG. 1A embodiment, a related art Mach-Zehnder interferometer system for OCT scanning is shown. FIG. 1B shows components for a related art Michelson interferometer system. In FIGS. 1A-1B, programmable filter 10 provides part of the laser cavity to generate tuned laser 50 output. The variable tuned laser 50 output goes through a coupler 38 and to a sample arm 40 and a reference arm 42. In FIG. 1A, the sample arm 40 signal goes through a circulator 44 and to a probe 46 for measurement of a sample S. The reference arm 42 signal is directed by a reference, which can be a mirror or a light guide, through a coupler 58 to a detector 60. The sampled signal is directed back through circulator 44 (FIG. 1A) and to a detector 60 through a coupler 58.

In FIG. 1B, the signal goes directly to sample arm 40 and reference arm 42; the sampled signal is directed back through coupler 38 and to detector 60. The detector 60 may use a pair of balanced photodetectors configured to cancel common mode noise.

As shown in FIGS. 1A and 1B, a control logic processor (control processing unit CPU) 70 is in signal communication with tuned laser 50 and its programmable filter 10 and with detector 60 and obtains and processes the output from detector 60. CPU 70 is also in signal communication with a display 72 for command entry and OCT results display.

FIG. 1C is a schematic diagram showing an OCT apparatus using a spectrometer 230 in a related art spectral domain (SD) OCT apparatus 240. A broadband source 224 directs light through coupler 38 to probe 46 for obtaining sampled scans of an intraoral feature or other subject. Scanning components that are part of probe 46 direct light from broadband illumination source 224 toward a plurality of points along the intraoral feature to perform the B-scan and C-scan. Low-coherence light from a broadband source 224 is directed through coupler 38 to probe 46 on sample arm 40 and to reference arm 42. The illumination source 224 can be a superluminescent diode, for example.

The interference pattern that is generated is measured at spectrometer 230. The light goes through a light dispersion optic 20 such as a grating, which provides dispersion of the light. Lens L2 optics then direct this light to a detection array 232. Detection array 232 can be a CCD (charge-coupled device) array or other sensor in the spectrometer that senses the selected wavelengths or wave-numbers. A processor 236, in signal communication with broadband source 224, spectrometer 230 and scanner then provides the logic and control circuitry for image reconstruction and display.

According to an alternate implementation of a generalized OCT apparatus, an arrangement offering Frequency-Modulated Continuous-Wave (FMCW) interferometry can be provided. FMCW interferometry allows the same probe (e.g., probe 46) to provide information for surface characterization of overall tooth, jaw, and facial structure as well as for characterization of particular surfaces for intraoral and extraoral features.

The schematic diagram of FIG. 1D shows components of a related art OCT apparatus 10 for acquiring FMCW data from a sample S. A tunable laser diode 320 provides a variable frequency monochromatic output light signal to coupler 14 components that provide a small portion of the light to an interferometer 16, which has fixed path length difference to provide an optical clock for the data acquisition, so that the acquired signal can be made linear in frequency. A second 1×2 coupler 14′ splits the remaining light along two paths of another interferometer 16: a sample path 24 and a reference local oscillator path 28. The light along the reference path 28 is directed to a 2×2 coupler 34 that provides the local oscillator signal to a balanced detector (B.D.) 30. Light along sample path 24 goes to a circulator 32 and from a scanner 22 to sample S. Returned light reflected from sample S goes back to circulator 32 and to 2×2 coupler 34, then on to balanced detector 30. A processor 36 obtains a range measurement according to the interference signals from sample and local oscillator reference paths 24 and 28 that are combined at coupler 34 and detected by detector 30.

Tunable laser source 320 is energizable to generate a light signal that is modulated in frequency. An exemplary tunable laser source is an external cavity diode laser from Thorlabs, Newton, NJ or a tunable pulse fiber from idealphotonics, Vancouver, Canada. The laser source can be based on Littrow or Littman model configurations. Other examples of tunable laser sources include distributed feedback lasers and tunable vertical cavity surface-emitting lasers.

The modulated light frequency from tunable laser source 320 can be swept in a linear progression and follows a sawtooth profile with respect to time. As the signal propagates through sample S, scattering and reflection direct a portion of the signal back to balanced detector 30 that detects interference between the returned signal from the sample and local oscillator signals. Alternately, the modulated frequency can have a triangular profile, or other suitable characteristic profile, with respect to time.

As shown in FIG. 1D, an optional demodulation and low pass filter 98 can be provided at the output signal from the balanced detector 30 for selectively acquiring only a portion of the detected data.

The simplified schematic diagram of FIG. 1E shows an optical arrangement for a related art Mach-Zehnder interferometer for FMCW imaging of a sample S. Light from tunable laser source 320 is split into local oscillator path 28 and sample path 24. A beam splitter BS1 is shown for directing light into the two paths 28 and 24. Mirrors M1 and M2 fold the optical path as needed for compactness in both sample and local oscillator paths 24, 28. Light from sample S and from local oscillator path 28 is combined by a second beam splitter BS2 in order to form an interference pattern that is sensed by detector 30, such as a balanced detection photodiode.

An implementation for intraoral OCT imaging apparatus 100 using FMCW interferometry can use any suitable interferometry model such as the related art Mach-Zehnder interferometer model shown in FIG. 1E or the related art Michelson interferometer as shown in FIG. 1F. In the FIG. 1F implementation, the sample path uses beam splitter BS1 to route the local oscillator and sample signal to and from sample S. In the Michelson arrangement of FIG. 1F, the signal goes directly to sample path 24 and local oscillator path 28; the sampled signal is directed back through beam splitter BS1 to detector 30.

Intra-oral OCT systems have been designed using related art interferometry instrumentation and techniques. In related art designs for OCT implementation, intraoral probe (e.g., probe 46) contains only the minimum of components needed in order to acquire the scan data; the interferometry and related processing are performed using components that are in signal communication with, but separate from, the probe (e.g., probe 46).

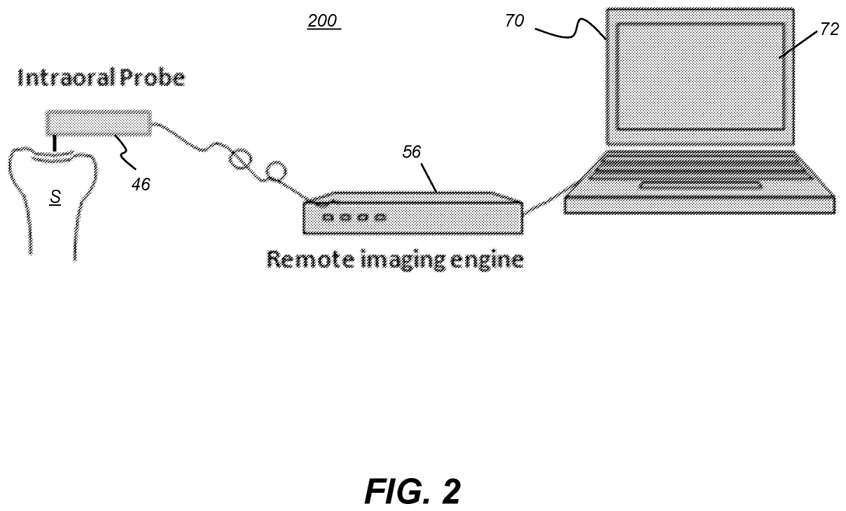

As shown in FIG. 2, related art OCT imaging system 200 includes probe 46 having the minimum of components needed in order to acquire the scan data for scanning sample S and an associated remote imaging engine 56 that includes the swept-source components, reference arm and fiber coupling optics, signal detectors, and acquisition and processing circuitry needed to provide intra-oral OCT imaging capabilities. Imaging engine 56 includes the light source, fiber coupler, reference arm, and OCT detector components described with reference to FIGS. 1A, 1B, 1C, 1D, 1E and 1F. Probe 46, in one embodiment, includes the raster scanner 90 or partial sample arm. Optional CPU 70 includes control logic and optional display 72 can display imaging results or dental applications. The requirement to keep probe 46 itself compact, lightweight, and manageable for operator scanning (e.g., handheld) of the patient dentition has thus far necessitated the use of a separate remote imaging engine 56 for support of the needed interferometry.

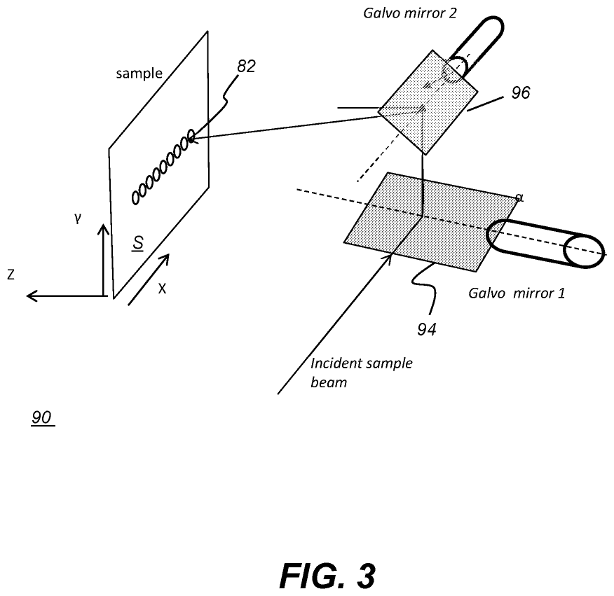

It is instructive to outline the scanning behavior used for OCT acquisition and to briefly review how OCT data is obtained. As shown in the schematic diagram of FIG. 3, galvo mirrors 94 and 96 cooperate to provide the raster scanning needed for related art OCT imaging. In the arrangement that is shown, galvo mirror 1 (94) scans the wavelengths of light to each point 82 along the sample to generate data along a row, in the x direction, which provides the B-scan, described in more detail subsequently. Scanning light for depth data is directed to sample S in the z-direction. Galvo mirror 2 (96) progressively moves the row position in the y direction to provide 2-D raster scanning to additional rows. At each point 82, the full spectrum of light provided is rapidly generated in a single sweep and the resulting signal measured at detector 60 (e.g., FIGS. 1A, 1B).

### Scanning Sequence for OCT Imaging

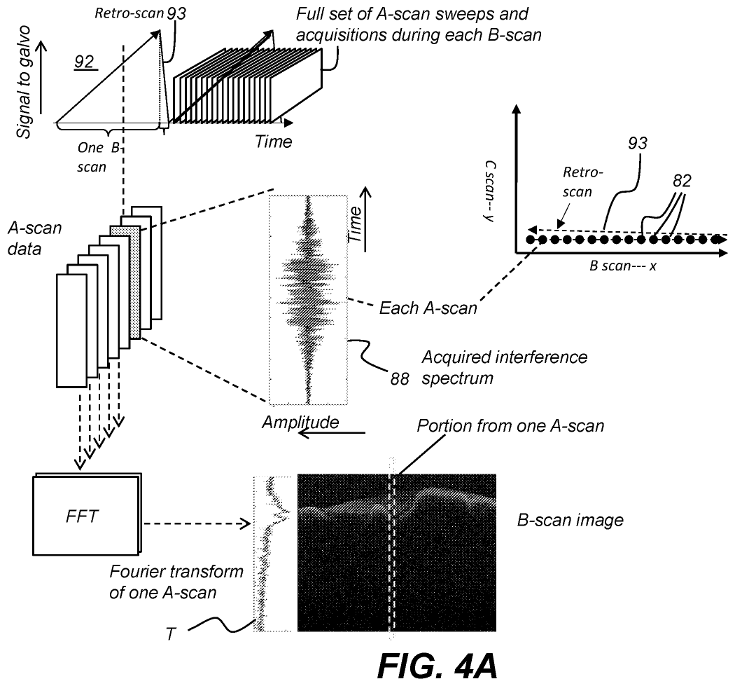

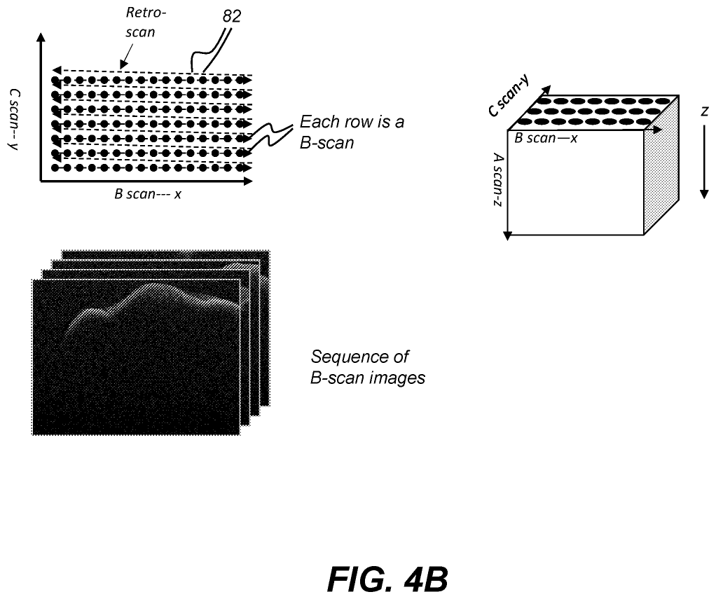

The schematic diagrams of FIGS. 4A and 4B show a scan sequence that can be used for forming tomographic images using example embodiments of OCT apparatus according to the present disclosure. The sequence shown in FIG. 4A shows how a single B-scan image is generated. A raster scanner 90 (FIG. 3) scans the selected light sequence over sample S, point by point. A periodic drive signal 92 as shown in FIG. 4A is used to drive the raster scanner 90 galvo mirrors to control a lateral scan or B-scan that extends across each row of the sample, shown as discrete points 82 extending in the horizontal direction in FIGS. 4A and 4B. At each of a plurality of points 82 along a line or row of the B-scan, an A-scan or depth scan, acquiring data in the z-axis direction, is generated using successive portions of the selected wavelength band. FIG. 4A shows drive signal 92 for generating a straightforward ascending sequence using raster scanner 90, with corresponding micro-mirror actuations, or other spatial light modulator pixel-by-pixel actuation, through the wavelength band. The retro-scan signal 93, part of drive signal 92, simply restores the scan mirror back to its starting position for the next line; no data is obtained during retro-scan signal 93.

It should be noted that the B-scan drive signal 92 drives the galvo mirror 94 for raster scanner 90 as shown in FIG. 3. At each incremental position, point 82 along the row of the B-scan, an A-scan is obtained. To acquire the A-scan data, tuned laser 50 or other programmable light source sweeps through the spectral sequence that is controlled by programmable filter 10. Thus, in an embodiment in which programmable filter 10 causes the light source to sweep through a 30 nm range of wavelengths, this sequence is carried out at each point 82 along the B-scan path. As FIG. 4A shows, the set of A-scan acquisitions executes at each point 82, that is, at each position of the scanning galvo mirror 94. By way of example, where a MEMs micro-mirror array device is used as a spatial light modulator, there can be 2048 measurements for generating the A-scan at each position 82.

FIG. 4A schematically shows the information acquired during each A-scan. An interference signal 88, shown with DC signal content removed, is acquired over the time interval for each point 82, wherein the signal is a function of the time interval required for the sweep, with the signal that is acquired indicative of the spectral interference fringes generated by combining the light from reference and feedback arms of the interferometer (e.g., FIGS. 1A, 1B). The Fourier transform generates a transform T for each A-scan. One transform signal corresponding to an A-scan is shown by way of example in FIG. 4A.

From the above description, it can be appreciated that a significant amount of data is acquired over a single B-scan sequence. In order to process this data efficiently, a Fast-Fourier Transform (FFT) is used, transforming the time-based signal data to corresponding frequency-based data from which image content can more readily be generated.

In Fourier domain OCT, the A scan corresponds to one line of spectrum acquisition which generates a line of depth (z-axis) resolved OCT signal. The B scan data generates a 2-D OCT image along the corresponding scanned line.

Raster scanning is used to obtain multiple B-scan data by incrementing the raster scanner 90 acquisition in the C-scan direction. This is represented schematically in FIG. 4B, which shows how 3-D volume information is generated using the A-, B-, and C-scan data.

As noted previously, the wavelength or frequency sweep sequence that is used at each A-scan point 82 can be modified from the ascending or descending wavelength sequence that is typically used. Arbitrary wavelength sequencing can alternately be used. In the case of arbitrary wavelength selection, which may be useful for some particular implementations of OCT, only a portion of the available wavelengths are provided as a result of each sweep. In arbitrary wavelength sequencing, each wavelength can be randomly selected, in arbitrary sequential order, to be used in the OCT system during a single sweep.

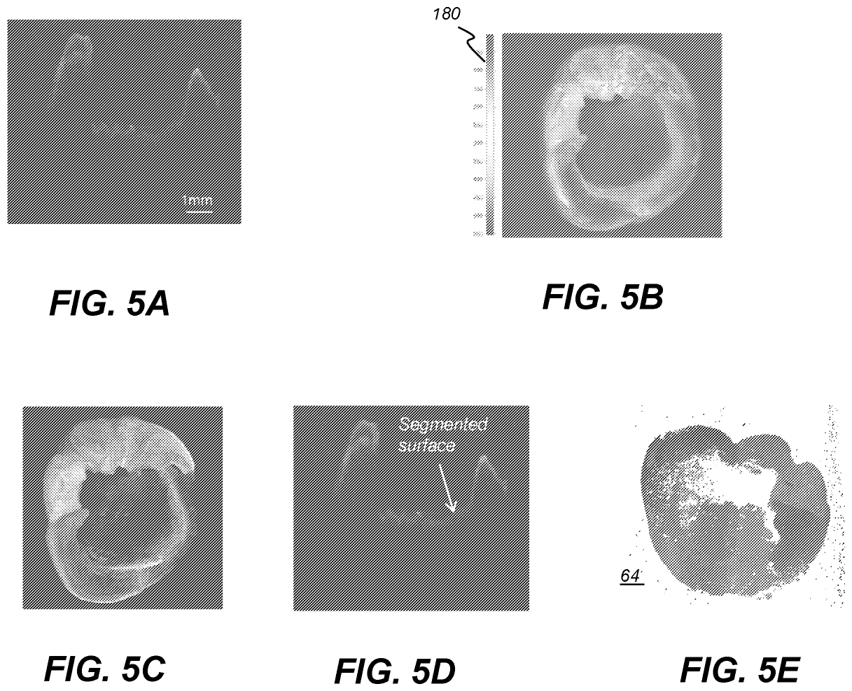

FIGS. 5A-5E show different types of imaging content acquired and generated as part of an OCT processing sequence, using the example of a tooth image having a severe cavity. FIG. 5A shows a 2-D slice that corresponds to a B-scan for OCT imaging. FIG. 5B shows a depth-encoded color projection of the tooth, with an optional color reference bar 180. FIG. 5C shows a corresponding slice of the volume rendering obtained from the OCT imaging content. FIG. 5D shows the results of segmentation processing of FIG. 5A in which points along the tooth surface are extracted. FIG. 5E shows a surface point cloud 64 of the tooth generated from the OCT volume data. The surface point cloud 64 can be obtained from the OCT volume data following segmentation.

Exemplary method and/or apparatus implementations according to the present disclosure provide embodiments of a handheld OCT scanner apparatus (e.g., 600, 700, 800, 900, 1000, 1100, 1200, 1300, 1400) that are highly compact and, by using photonic integrated circuits, reduce or eliminate the need for at least some portion of the external equipment (e.g., provided in remote imaging engine 56) needed for related art intraoral OCT apparatus configurations described to date.

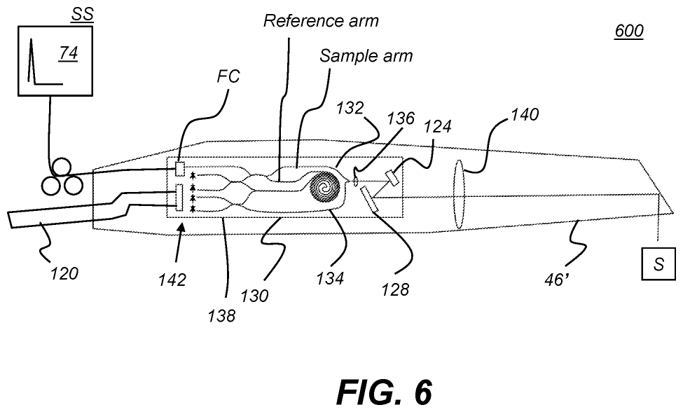

FIG. 6 shows an example embodiment for a handheld OCT scanner apparatus using photonic integrated circuits in accordance with some implementations. Referring to the schematic diagram of FIG. 6, probe 46′ of a handheld OCT scanner apparatus 600 is shown connected to an external laser light source 74 that provides a swept-source laser signal that sweeps through a range of wavelengths for OCT data collection.

In the FIG. 6 embodiment, a fiber coupler FC couples the laser light to the source arm of an interferometer 130 that is formed as a photonic integrated circuit on a substrate 138 (e.g., a silicon substrate) and built into a housing (e.g., handle) of probe 46′. Internal output waveguide 132 and collection waveguide 134 are etched onto the same silicon substrate 138. Light in the waveguide structure, from a swept-source SS, is split into a sample arm and a reference arm. The sample light is coupled to the output and collimated by a micro collimation lens 136. The collimated light is focused onto the sample S by an objective lens 140 that is optimized for imaging. The focused beam is steered from a folding mirror 124 onto the sample S by a MEMS mirror 128 that is coupled to the interferometer 130. Lens 136, mirror 124, and MEMS mirror 128 are components that can also be integrated on silicon substrate 138 as shown in FIG. 6.

Collection waveguide 134 is located in proximity to (e.g., several microns from, such as 100 microns from) the output waveguide 132 for collecting backscattered light from the sample. The output waveguide 132 also collects some amount of backscattered light. The reference arm path length matches the sample arm path length. The reference light is split into two reference light parts and interferes with the first and second sample light. Interference fringes are detected by two sets of on-chip balanced photo detectors 142. Each one of the two sets of balanced photo detection provides the complete interferometric signal for OCT reconstruction. Thus, it is optional to have both sets of balanced photo detectors; however, having both sets increases the signal to noise ratio.

A data bus 120 connects to probe 46′ and may include power connection, which can alternately be provided separately.

For intraoral OCT, for example, laser 50 can be tunable over a range of frequencies (wave-numbers k) corresponding to wavelengths between about 400 and 1600 nm. According to an example embodiment of the present disclosure, a tunable range of about 60 nm bandwidth centered about 1300 nm is used for intraoral OCT.

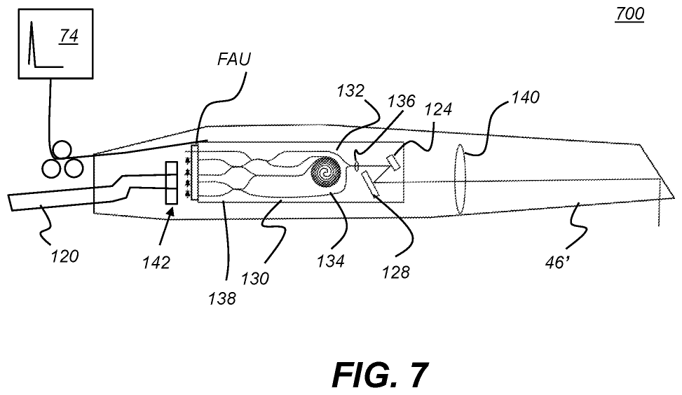

FIG. 7 shows an example embodiment for a handheld OCT scanner apparatus using photonic integrated circuits in accordance with some implementations. The embodiment of FIG. 7 is similar to that of FIG. 6, but employs a fiber array unit (FAU) in a handheld OCT scanner apparatus 700. In addition, balanced photo-detectors 142 are positioned separately from the silicon substrate 138 used for the interferometer. The balanced photo-detectors 142 are within a housing of the apparatus 700. A fiber array unit FAU is used to couple the laser light to the interferometer 130 that is formed as a photonic integrated circuit.

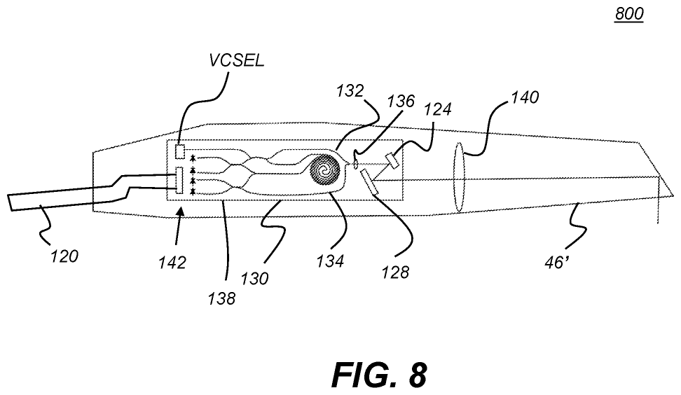

FIG. 8 shows an example embodiment for a handheld OCT scanner apparatus using photonic integrated circuits in accordance with some implementations. The embodiment of FIG. 8 is similar to that of FIG. 6 but has the swept-source light source integrated on the silicon substrate 138 used for interferometer 130 in a handheld OCT scanner apparatus 800. The integrated swept-source can be a wavelength tunable vertical-cavity surface-emitting laser (VCSEL) with micromechanically movable mirrors. The VCSEL can be optically or electrically pumped. Alternately, the integrated swept-source can be a monolithic semiconductor swept-source laser.

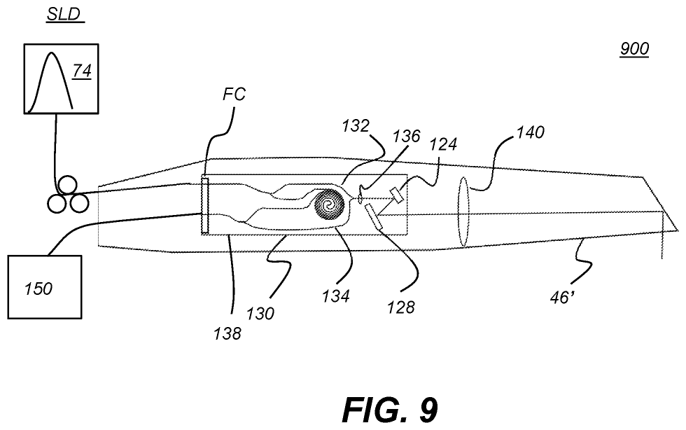

FIG. 9 shows an example embodiment for a handheld OCT scanner apparatus using photonic integrated circuits in accordance with some implementations. The FIG. 9 embodiment shows a spectral domain (SD) OCT configuration using an external SLD (Super-Luminescent Diode) as light source 74 in a handheld OCT scanner apparatus 900. A separate spectrometer 150 is also provided to detect the interferometer signal, external to probe 46′.

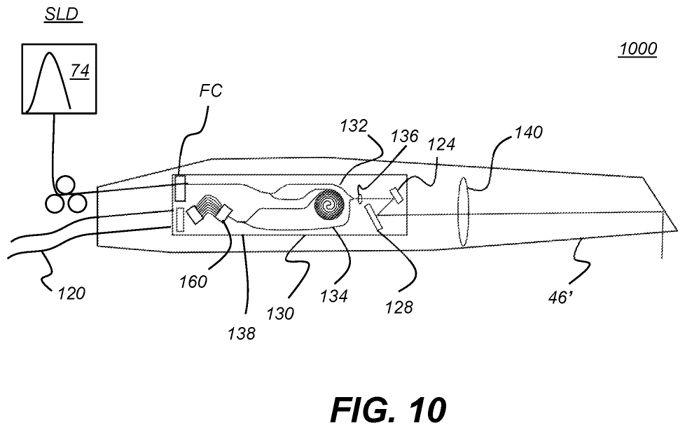

FIG. 10 shows an example embodiment for a handheld OCT scanner apparatus using photonic integrated circuits in accordance with some implementations. The embodiment of FIG. 10 employs an integrated spectrometer 160 that is formed on the silicon substrate 138 of the interferometer 130 in a handheld OCT scanner apparatus 1000. This can be a spectrometer 160 that uses an arrayed waveguide grating, for example.

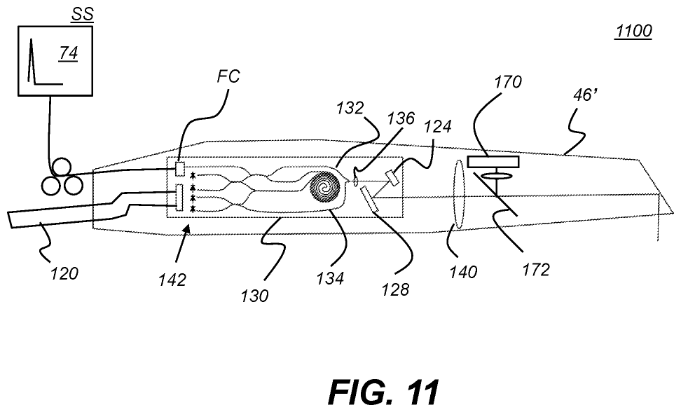

FIG. 11 shows an example embodiment for a handheld OCT scanner apparatus using photonic integrated circuits in accordance with some implementations. The embodiment of FIG. 11 shows probe 46′ for OCT scanning with additional components for reflectance imaging in a handheld OCT scanner apparatus 1100. An image acquisition apparatus 170 can be provided, configured to use portions of the same optical path for reflectance and OCT scanning using a beam combiner 172, such as a dichroic mirror. Image acquisition apparatus 170 can include the necessary optics, light source, and image sensing components for monochrome or color imaging, such as for providing a preview image to the operator. Optionally, image sensing apparatus 170 can also include components that provide structured light projection and acquisition for surface contour imaging. Reflectance imaging can use light in the visible or near-visible range, such as near-IR light for example.

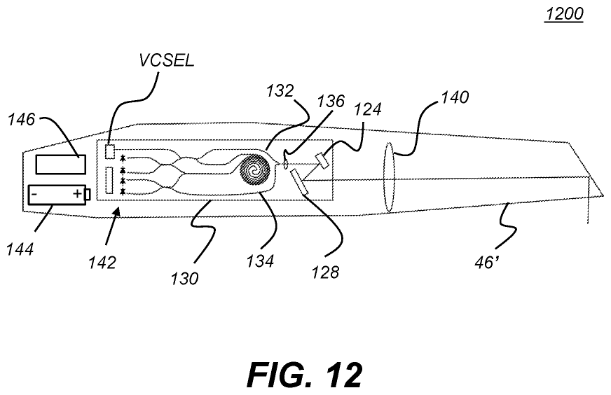

FIG. 12 shows an example embodiment for a handheld OCT scanner apparatus using photonic integrated circuits in accordance with some implementations. According to an alternate embodiment, as shown in the schematic of FIG. 12, probe 46′ of a handheld OCT scanner apparatus 1200 can include a battery 144 or other replaceable or rechargeable power source. As shown in FIG. 12, probe 46′ can also have a transmitter 146 for wireless communication and data transfer with a host processor.

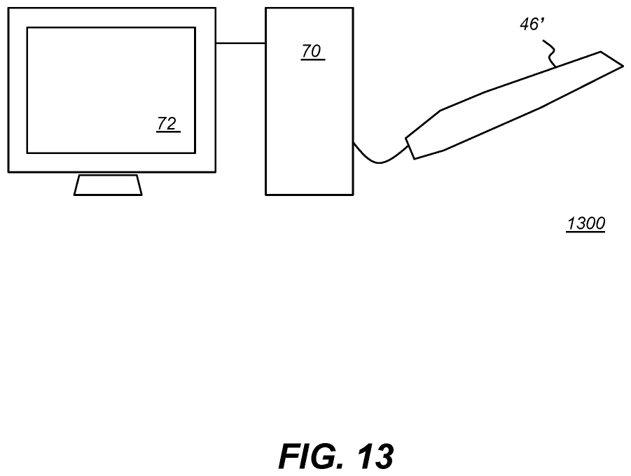

FIG. 13 shows an example embodiment for a handheld OCT scanner apparatus using photonic integrated circuits in accordance with some implementations. The schematic diagram of FIG. 13 shows an intraoral OCT imaging apparatus 1300 using probe 46′ according to a tethered configuration. Control logic processor 70 and display 72 can be incorporated into a single unit, such as a laptop computer, for example. In FIG. 13, control logic processor 70 can alternately be provided as embedded electronics, formed within probe 46′.

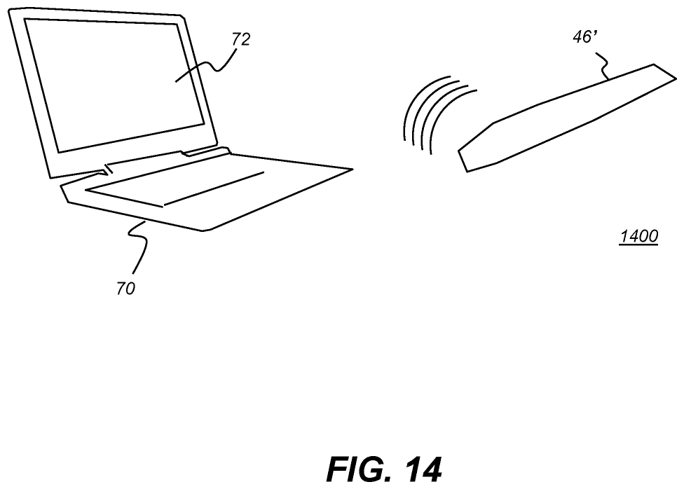

FIG. 14 shows an example embodiment for a handheld OCT scanner apparatus using photonic integrated circuits in accordance with some implementations. The schematic diagram of FIG. 14 shows an intraoral OCT imaging apparatus 1400 using probe 46′ according to an un-tethered configuration that employs wireless communication.

Consistent with an example embodiment, a computer program utilizes stored instructions that perform on image data that is accessed from an electronic memory. As can be appreciated by those skilled in the image processing arts, a computer program for operating the imaging system in an example embodiment of the present disclosure can be utilized by a suitable, general-purpose computer system operating as a CPU as described herein, such as a personal computer or workstation. However, many other types of computer systems can be used to execute the computer program of the present invention, including an arrangement of networked processors, for example. The computer program for performing example methods of the present invention may be stored in a computer readable storage medium. This medium may comprise, for example; magnetic storage media such as a magnetic disk such as a hard drive or removable device or magnetic tape; optical storage media such as an optical disc, optical tape, or machine readable optical encoding; solid state electronic storage devices such as random access memory (RAM), or read only memory (ROM); or any other physical device or medium employed to store a computer program. The computer program for performing example methods of the present disclosure may also be stored on computer readable storage medium that is connected to the image processor by way of the internet or other network or communication medium. Those skilled in the art will further readily recognize that the equivalent of such a computer program product may also be constructed in hardware.

It should be noted that the term “memory”, equivalent to “computer-accessible memory” in the context of the present disclosure, can refer to any type of temporary or more enduring data storage workspace used for storing and operating upon image data and accessible to a computer system, including a database, for example. The memory could be non-volatile, using, for example, a long-term storage medium such as magnetic or optical storage. Alternately, the memory could be of a more volatile nature, using an electronic circuit, such as random-access memory (RAM) that is used as a temporary buffer or workspace by a microprocessor or other control logic processor device. Display data, for example, is typically stored in a temporary storage buffer that is directly associated with a display device and is periodically refreshed as needed in order to provide displayed data. This temporary storage buffer is also considered to be a type of memory, as the term is used in the present disclosure. Memory is also used as the data workspace for executing and storing intermediate and final results of calculations and other processing. Computer-accessible memory can be volatile, non-volatile, or a hybrid combination of volatile and non-volatile types.

It will be understood that example computer program products of the present disclosure may make use of various image manipulation algorithms and processes that are well known. It will be further understood that example computer program product embodiments of the present disclosure may embody algorithms and processes not specifically shown or described herein that are useful for implementation. Such algorithms and processes may include conventional utilities that are within the ordinary skill of the image processing arts. Additional aspects of such algorithms and systems, and hardware and/or software for producing and otherwise processing the images or co-operating with example computer program products of the present disclosure, are not specifically shown or described herein and may be selected from such algorithms, systems, hardware, components and elements known in the art.

In accordance with some implementations, a device includes one or more processors, a non-transitory memory, and one or more programs; the one or more programs are stored in the non-transitory memory and configured to be executed by the one or more processors and the one or more programs include instructions for performing or causing performance of any of the methods described herein. In accordance with some implementations, a non-transitory computer readable storage medium has stored therein instructions, which, when executed by one or more processors of a device, cause the device to perform or cause performance of any of the methods described herein. In accordance with some implementations, a device includes: one or more processors, a non-transitory memory, and means for performing or causing performance of any of the methods described herein.

In various implementations, example embodiments of an intraoral handheld optical apparatus include a housing, an interferometer with at least output and collection waveguides formed on a photonic integrated circuit substrate, where the photonic integrated circuit substrate is within the housing, a light source configured to generate light of wavelengths above a threshold wavelength, a first signal detector configured to obtain an interference signal from the interferometer between a first portion of the light scattered from the sample and a reference portion of the light, and a processor to perform optical coherence tomography processing on the obtained interference signal.

In some example embodiments, the light source is within the housing or the light source is formed on the photonic integrated circuit substrate. In some example embodiments, the first signal detector is within the housing or the first signal detector is formed on the photonic integrated circuit substrate.

In some example embodiments, the photonic integrated circuit substrate includes a microelectromechanical systems scanning mirror. In some example embodiments, a second signal detector is configured to obtain an interference signal from the interferometer between a second portion of the light scattered from the sample and a reference portion of the light. In some example embodiments, the first portion of the light scattered from the sample goes through the collection waveguide and the second portion of the light scattered from the sample goes through the output waveguide. In some example embodiments, the second signal detector is formed on the photonic integrated circuit substrate.

In some example embodiments, a wireless transmitter or battery is within the housing. In some example embodiments, the processor can perform frequency-modulated continuous wave (FMCW) processing on the obtained interference signal. In some example embodiments, the processor is within the housing. In some example embodiments, the housing includes a second light source to emit light in the visible range, a beam combiner lies in the path of the light to and from the sample and in the path of light from the second light source, and an image acquisition apparatus to obtain reflectance image data from the sample. In some example embodiments, the sample is an intraoral feature of a patient.

Exemplary implementations according to the application can include various features described herein (individually or in combination).

While the invention has been illustrated with respect to one or more implementations, alterations and/or modifications can be made to the illustrated examples without departing from the spirit and scope of the appended claims. The presently disclosed implementations are therefore considered in all respects to be illustrative and not restrictive. In addition, while a particular feature of the invention can have been disclosed with respect to one of several implementations, such feature can be combined with one or more other features of the other implementations as can be desired and advantageous for any given or particular function. The term “at least one of” is used to mean one or more of the listed items can be selected. The term “about” indicates that the value listed can be somewhat altered, as long as the alteration does not result in nonconformance of the process or structure to the illustrated implementation. Other implementations of the invention will be apparent to those skilled in the art from consideration of the specification and practice of the invention disclosed herein. It is intended that the specification and examples be considered as exemplary only, with a true scope and spirit of the invention being indicated by the following claims, and all changes that come within the meaning and range of equivalents thereof are intended to be embraced therein.

1. A handheld optical apparatus for imaging a sample, the apparatus comprising:
a housing;
an interferometer having at least an output waveguide and a collection waveguide, wherein the output waveguide and the collection waveguide are formed on a photonic integrated circuit substrate, where the photonic integrated circuit substrate is within the housing;
a light source configured to generate light of wavelengths above a threshold wavelength;
a first signal detector configured to obtain an interference signal from the interferometer between a first portion of the light scattered from the sample and a reference portion of the light; and
a processor that is programmed with instructions to perform optical coherence tomography processing on the interference signal.

2. The apparatus of claim 1, where the light source is a superluminescent diode, where the light source is a wavelength tunable laser, where the light source is a wavelength tunable vertical-cavity surface-emitting laser, where the light source is an external cavity diode laser, or where the light source has a tunable range of about 60 nm bandwidth centered about 1300 nm.

3. The handheld optical apparatus of claim 1, where the light source is within the housing.

4. The handheld optical apparatus of claim 1, where the light source is formed on the photonic integrated circuit substrate.

5. The handheld optical apparatus of claim 1, where the first signal detector is a spectrometer

6. The handheld optical apparatus of claim 1, where the first signal detector is a balanced detector.

7. The handheld optical apparatus of claim 1, where the first signal detector is within the housing.

8. The handheld optical apparatus of claim 1, where the first signal detector is formed on the photonic integrated circuit substrate.

9. The handheld optical apparatus of claim 1, where the photonic integrated circuit substrate further comprises a microelectromechanical systems scanning mirror.

10. The handheld optical apparatus of claim 1, where the output waveguide and the collection waveguide are formed in proximity to each other for collecting the light scattered from the sample.

11. The handheld optical apparatus of claim 1, further comprising a second signal detector configured to obtain a second interference signal from the interferometer between a second portion of the light scattered from the sample and a reference portion of the light.

12. The handheld optical apparatus of claim 11, where the first portion of the light scattered from the sample goes through the collection waveguide and the second portion of the light scattered from the sample goes through the output waveguide.

13. The handheld optical apparatus of claim 11, where the second signal detector is formed on the photonic integrated circuit substrate.

14. The handheld optical apparatus of claim 1, further comprising a wireless transmitter that is configured to provide optical coherence tomography or frequency-modulated continuous wave (FMCW) data, where the wireless transmitter is within the housing.

15. The handheld optical apparatus of claim 1, further comprising a battery, where the battery is within the housing.

16. The handheld optical apparatus of claim 1, where the processor is programmed with instructions that perform frequency-modulated continuous wave (FMCW) processing on the interference signal.

17. The handheld optical apparatus of claim 1, where the processor is formed from embedded electronics, where the processor is within the housing.

18. The handheld optical apparatus of claim 1, further comprising a beam combiner and a reflectance image detector within the housing.

19. The handheld optical apparatus of claim 18, where the light source is a first light source, and further comprising:
a second light source to emit light in a visible range within the housing;
where the beam combiner lies in a first path of the light to and from the sample and in a second path of light from the second light source; and
an image acquisition apparatus to obtain reflectance image data from the sample.

20. The handheld optical apparatus of claim 1, where the sample is an intraoral feature of a patient.
